SEQUENCE LISTING (9 sequences)

SEQ ID NO: 1 - DNA, 5922 bp, Homo sapiens
  CDS (348)..(4244)
gcggccgcggggaccctcggcgtggtcctctgaccctgcaaacccgcgacggaggaaggggaggtcctgcccgaggcgccagcccgaggaggaggatgcccatttaacccgccctcgcctgccgggcgcttgcctcggtgcccgccgccggagcctccgagccgcgcccgtggaagtgctgcatggggcagggctgctgaagcgcggagttcggggtcgcgccgctcccaggcaggcgcgggagcccggtgcggcagttggcacagtttcggcggcgccttctgcgcgggagtggggggcgcggtgcgcccggccggcctccgcggtgccctggtgaggcgagagttatggagccgcccagctgcattcaggatgagccgttcccgcaccccctggagcccgagccgggcgtctcagctcagcccggccccgggaagccaagcgataagcggttccggctgtggtacgttggggggtcgtgcctggaccacaggaccacgctgcctatgctgccctggctcatggccgagatccgcaggcgcagccagaagcccgaggcgggcggctgcggggcgccggcggcccgagaggtgatcctggtgctcagcgcgcccttcctgcgttgcgtccccgcgccgggcgctggggcctcggggggcactagtccgtcggccacgcagcccaacccggcggtattcatcttcgagcacaaggcgcagcatatctcgcgcttcatccacaacagccacgacctcacctactttgcctacctgatcaaggcgcagcccgacgaccccgagtcgcagatggcctgccacgttttccgcgccacagaccccagccaggttcctgatgttattagcagcataaggcaattatctaaagcggccatgaaagaggatgccaaacccagcaaagataatgaggacgccttttacaactctcagaagttcgaagtcctgtactgtggaaaggtgaccgtgacccacaagaaggccccctcaagcctcatcgatgactgcatggagaagttcagcctgcacgaacagcagcgcctgaagatccaaggcgagcagcgcggtccggacccaggagaggacctggctgacttggaggtggtggtgcccgggtcccccggagactgcctgccggaggaggctgacggcaccgacacccaccttggcttacctgccggggccagccagcctgccctgaccagctctcgggtctgcttccctgagcggattttggaagattctggctttgatgagcagcaggagtttcggtctcggtgcagcagtgtcaccggcgtgcaacggagagttcacgagggcagccagaaatcccagccgcgacggagacacgcgagcgcacccagtcacgtccagccctcggactcggagaagaacaggaccatgctcttccaggttgggcgatttgagattaaccttatcagtccagacactaaatcagttgtgctagaaaagaattttaaagatatctcctcttgttctcagggtataaagcatgtggatcactttggctttatctgccgggagtctccagagcctggacttagccagtatatttgttatgtattccagtgtgccagcgaatctctggttgatgaggtaatgctgactctgaaacaggccttcagtacggcggctgccctgcagagtgccaagacgcagattaaactgtgtgaggcctgcccgatgcactctttgcataagctctgtgaaaggattgaaggtctctacccaccaagagccaagctggtgatacagaggcatctctcatcactgacagataatgagcaagctgacatctttgaaagagttcagaaaatgaagccagtcagtgaccaggaagaaaatgaacttgtgattttacacctgaggcagctgtgtgaagccaagcagaagacacacgtgcacatcggggaaggcccttctactatttcaaatagtacaatcccagaaaatgcaacaagcagtggaaggttcaaacttgacattctgaaaaataaagctaagagatccttaactagctccctggaaaatatcttctcaaggggagctaacagaatgagaggtcggcttggaagtgtggacagttttgaacggtccaacagtcttgcttcagagaaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcagcaatctttcgtcagttcgacgcatgtacaaggagagtaattcttcctccagtcttccaagtcttcacacttccttctctgccccttccttcactgccccctctttcctgaaaagcttttaccagaattcaggtagactgtccccacagtatgaaaatgaaatcagacaagacactgcttcagaatcaagtgatggagaagggagaaaaaggacctcatctacctgcagcaatgagtccctaagtgtgggaggaacctctgtcactcctcgccggatctcctggcggcagcgcattttcctcagggttgcttctcccatgaacaaatctccctcagcaatgcaacagcaagatggattggacaggaacgagctgctgccactgtcccccctctctccaaccatggaggaggaaccgctggttatattcctgtctggggaggatgacccagaaaagattgaagaaagaaagaaatcaaaagaactgaggagcttgtggagaaaagctatacaccaacaaatcttgttacttcgaatggaaaaagaaaaccagaaacttgaaggagcaagcagagatgaactccagtccagaaaagttaaattagactatgaagaagttggtgcatgtcagaaagaggtcttaataacttgggataagaagttgttaaactgcagagctaaaatcagatgtgatatggaagatattcatactcttcttaaagaaggagttcccaaaagtcgacgaggagaaatttggcagtttctggctttacagtaccgactcagacacagattgcctaataaacaacagcctcctgacatatcctataaggaacttttgaagcagctcactgctcagcagcatgcgattctcgtggatttaggaaggacgtttcctactcacccttacttttcagtacagcttgggccaggacagctgtcactgtttaacctcctgaaagcctattctttgctggacaaagaagtgggatactgtcaggggatcagctttgtggctggagtcctgcttctgcacatgagtgaagagcaagcctttgaaatgctgaaattcctcatgtatgacctcggcttccgcaagcagtacagacctgacatgatgtcgctgcagattcaaatgtaccagctgtccaggctccttcatgactatcacagagatctctacaatcaccttgaagaaaatgaaatcagccccagtctttatgctgccccctggttcctcacattgtttgcctctcagttttcattaggatttgtagccagagtttttgatattatttttcttcagggaactgaagttatattcaaggttgcactcagcctactgagcagccaagagacacttataatggaatgtgagagctttgaaaatattgttgagtttcttaaaaacacgctacctgatatgaatacctctgaaatggaaaaaattattacccaggtttttgagatggatatttctaagcagttgcatgcctatgaggtggaatatcatgtgctacaggatgagcttcaggaatcttcatattcctgtgaggatagtgaaactttggagaagctggagagggccaatagccaactgaaaagacaaaacatggacctcctagaaaaattacaggtagctcatactaaaatccaggccttggaatcaaacctggaaaatcttttgacgagagagaccaaaatgaagtctttaatccggaccctggaacaagaaaaaatggcttatcaaaagacagtggagcaactccggaagctgctgcccgcggatgctctagccaattgtgacctgttgctgagagacctaaactgcaaccctaacaacaaagccaagataggaaataagccataattgaagaggcacggcctcagcagaaagtgctccttagaatactacagagaggaagagcctgcatgtcgctggcccaaggctggaccctgaagctgatggaaccacctaatactggtgctgagctcctagtcacagcaggtggacctcgtgctcatcagagcatgccaatcctaagccattggacatatgtagactggtttttgttgttgctatgtacatataaatatatatataaaatgaacatagttcatgctttcagataaaatgagtagatgtatatttagattaatttttttagtcagaacttcatgaaatccacaccaaaggaaaggtaaactgaaatttcccttggacatatgtgaaatctttttgtctttatagtgaaacaaagccagagcatctttgtatattgcaatatacttgaaaaaaatgaatgtatttttttctccaaagaacagcatgtttcactcaatggtgaaaaggtggaaacatttatgtaactttatgtgtatctgtcttgatatctactgacattgtctatatgaggaaaatgattactggtcatgctcctgtgagttttttgggaaggtagggtcatttctccctgcctgctttgtgccaactagcatgttgcatctacatgcattatgagtctggttaggcattactttaaacatacataaagagacagtaggacattgtggctgagtctacccagctcaaggtaaaggagaatgttgctaattttttagcaaactagaccagcattattactcaaactaaaaatatcacacctgaaaaatttaatttaggacctaaaatgtctagattagctttctgctttttttatttgaataactcattcagttgtgaatgaattcctctttatttggtgccacagtcaccaaatgacaaggatttgccactttcccaccaaattgtgagtgcttgtaatttaggtctctctaccttaaattcagtataaggaaacgtaattatgattgattttttccaaagatgacaagctgtgttgaaatacattttttcttttgaccaattgacagaatctaataagctttaataatcttccccttttatgtgaaaagttttgagaactgtgaaatgtttaggaacaaactgttgaaatccattggaagggaaaaaagaaagtggtaccagtgttaccagctcaactaaaacctgcaattctgcatttcaactcttcacttcctcagcctacaaatagctcattagatgacattcacgcatgctgggtataggcaaggaaagtaattttcaaagtacatttgcagttctctttttcagagatgattctatgatagtgcctctgaaagttgatgcagcatttttgcctttccaaaaagtatttatcctcactgctttttgcagtacttgtattttcacagatggattatctggggtaattttcttcaaagggagtttgttatacacagtgaaaatgtattatagagtagaatagtaaagctctaggggtttcagaaagctttgatgaacagatgacaaacatctgaaaccccctccgcactgttacccagtgtgtatataatgacttgttatagctcagtgtgcccttgaatccatacagtttcttaaaagacaataaaatcttattaataaagttaatgtaacttct

SEQ ID NO: 2 - protein, 1299 aa, Homo sapiens
MEPPSCIQDEPFPHPLEPEPGVSAQPGPGKPSDKRFRLWYVGGSCLDHRTTLPMLPWLMAEIRRRSQKPEAGGCGAPAAREVILVLSAPFLRCVPAPGAGASGGTSPSATQPNPAVFIFEHKAQHISRFIHNSHDLTYFAYLIKAQPDDPESQMACHVFRATDPSQVPDVISSIRQLSKAAMKEDAKPSKDNEDAFYNSQKFEVLYCGKVTVTHKKAPSSLIDDCMEKFSLHEQQRLKIQGEQRGPDPGEDLADLEVVVPGSPGDCLPEEADGTDTHLGLPAGASQPALTSSRVCFPERILEDSGFDEQQEFRSRCSSVTGVQRRVHEGSQKSQPRRRHASAPSHVQPSDSEKNRTMLFQVGRFEINLISPDTKSVVLEKNFKDISSCSQGIKHVDHFGFICRESPEPGLSQYICYVFQCASESLVDEVMLTLKQAFSTAAALQSAKTQIKLCEACPMHSLHKLCERIEGLYPPRAKLVIQRHLSSLTDNEQADIFERVQKMKPVSDQEENELVILHLRQLCEAKQKTHVHIGEGPSTISNSTIPENATSSGRFKLDILKNKAKRSLTSSLENIFSRGANRMRGRLGSVDSFERSNSLASEKDYSPGDSPPGTPPASPPSSAWQTFPEEDSDSPQFRRRAHTFSHPPSSTKRKLNLQDGRAQGVRSPLLRQSSSEQCSNLSSVRRMYKESNSSSSLPSLHTSFSAPSFTAPSFLKSFYQNSGRLSPQYENEIRQDTASESSDGEGRKRTSSTCSNESLSVGGTSVTPRRISWRQRIFLRVASPMNKSPSAMQQQDGLDRNELLPLSPLSPTMEEEPLVIFLSGEDDPEKIEERKKSKELRSLWRKAIHQQILLLRMEKENQKLEGASRDELQSRKVKLDYEEVGACQKEVLITWDKKLLNCRAKIRCDMEDIHTLLKEGVPKSRRGEIWQFLALQYRLRHRLPNKQQPPDISYKELLKQLTAQQHAILVDLGRTFPTHPYFSVQLGPGQLSLFNLLKAYSLLDKEVGYCQGISFVAGVLLLHMSEEQAFEMLKFLMYDLGFRKQYRPDMMSLQIQMYQLSRLLHDYHRDLYNHLEENEISPSLYAAPWFLTLFASQFSLGFVARVFDIIFLQGTEVIFKVALSLLSSQETLIMECESFENIVEFLKNTLPDMNTSEMEKIITQVFEMDISKQLHAYEVEYHVLQDELQESSYSCEDSETLEKLERANSQLKRQNMDLLEKLQVAHTKIQALESNLENLLTRETKMKSLIRTLEQEKMAYQKTVEQLRKLLPADALANCDLLLRDLNCNPNNKAKIGNKP

SEQ ID NO: 3 - DNA, 164 bp, Homo sapiens
caaggtggattgtgaatttatgctgtagccaacttttagttttgagaaacaccataaaaacaaattaagttacctcatttactaggcgaaacaggcaaggttaaggcatacacaaaaagaagagttaattcgtttgggtggaaactcttttgtttttcctttca

SEQ ID NO: 4 - DNA, 486 bp, Homo sapiens
aaagggctcccttccgcttgtgtttcaggactactcaccaggggattctccaccagggacaccgccagcgtccccaccgtcctcagcttggcaaacgtttcccgaagaggattccgactccccgcagtttcgaagacgggcacacacgttcagccacccaccttcaagcacaaagagaaagctgaatttgcaggatgggagggctcagggtgtgcgttcccctctgctgaggcagagctccagtgaacagtgcaggtgagtctgaccctctccagaatgagacctagattctcaaggcacacagttttgattactctggagtcaaggtggattgtgaatttatgctgtagccaacttttagttttgagaaacaccataaaaacaaattaagttacctcatttactaggcgaaacaggcaaggttaaggcatacacaaaaagaagagttaattcgtttgggtggaaactcttttgtttttcctttca

SEQ ID NO: 5 - DNA, 17 bp, Artificial Sequence
  Synthetic
gtttttttttttttttc

SEQ ID NO: 6 - DNA, 10 bp, Artificial Sequence
  Synthetic
tctctggagt

SEQ ID NO: 7 - DNA, 21 bp, Artificial Sequence
  Synthetic
aagacagtggagcaactccgg

SEQ ID NO: 8 - DNA, 21 bp, Artificial Sequence
  Synthetic
cagcaacaggtcacaattggc

SEQ ID NO: 9 - DNA, 20 bp, Artificial Sequence
  Synthetic
agctgctgcccgcggatgct

(19) United States
(12) Patent Application Publication
(10) Pub. No.: US 2003/0224423 A1
(43) Pub. Date: Dec. 4, 2003

(54) Method of testing for allergic diseases

(75) Inventors: Yoshiko Matsumoto, Kawasaki-shi (JP); Yukiho Imai, Kawasaki-shi (JP); Nei Yoshida, Kawasaki-shi (JP); Tadahiro Oshida, Kawasaki-shi (JP); Yuji Sugita, Kawasaki-shi (JP); Hirohisa Saito, Tokyo (JP)

Correspondence Address: Peter G. Carroll, MEDLEN & CARROLL, LLP, Suite 350, 101 Howard Street, San Francisco, CA, 94105, US

(73) Assignees: Genox Research, Inc.; Japan as Represented by General Director of Agency of National Center for Child Health & Development

(21) Appl. No.: 10/408,025

(22) Filed: Apr. 3, 2003

(30) Foreign Application Priority Data
Apr. 3, 2002 (JP) 2002-100908
Feb. 25, 2003 (WO) PCT/JP03/02047

(51) Int. Cl. C12Q 1/68; G01N 33/567

(52) U.S. Cl. 435/6; 435/7.21

(57) ABSTRACT

B1799 gene, whose expression level increases significantly in the patient group, was successfully identified by screening for a gene which shows difference in expression in blood collected from multiple healthy subjects and patients of allergic diseases employing the differential display method. The gene expression level is specifically high in T cells and increased due to T cell activation. This gene can be used in testing for allergic diseases and also in screening for therapeutic agents for allergic diseases.

### FIELD OF THE INVENTION

The present invention relates to a method of testing for an allergic disease and a method of screening for a therapeutic agent for an allergic disease.

### BACKGROUND OF THE INVENTION

Allergic diseases such as atopic dermatitis are considered to be multifactorial diseases. These diseases are caused by the interaction of many different genes whose expression is independently influenced by multiple environmental factors. Therefore, it is extremely difficult to identify a specific gene causing a specific disease. Furthermore, the expression of mutated or defective genes, or increased or decreased expression of specific genes is envisaged to be associated with allergic diseases. Thus, to reveal the role of gene expression in diseases, it is required to understand how a gene is involved at the onset of a disease and how external stimulus, such as drugs, alters gene expression.

History taking as well as confirmation of the patient's family history and own anamnesis, are important in general for recent diagnosis of allergic diseases. In addition, for allergy diagnosis based on more objective information, a method of testing patient's blood samples and a method of observing patient's immune response to allergen are also performed. Examples of the former method are the allergen-specific IgE test, leukocyte histamine release test and lymphocyte blastogenesis test. The presence of allergen-specific IgE is proof of occurrence of the allergic reaction to the allergen. However, in some patients, allergen-specific IgE may not necessarily be detected. Furthermore, the IgE assay principle requires performing tests for all of the allergens necessary for diagnosis. The leukocyte histamine release test and the lymphocyte blastogenesis test are methods for observing the immune system reaction to allergens in vitro. These methods are complicated to perform.

Another known method is allergy diagnosis based on the immune response observed at the time when a patient is contacted with an allergen. Such a test includes the prick test, scratch test, patch test, intradermal reaction and induction test. These tests allow the direct diagnosis of patient's allergic reaction while they can be said to be highly invasive tests because patients are actually exposed to allergens.

In addition, test methods for proving the occurrence of allergic reaction caused by any allergen are also attempted. For example, a high serum IgE titer may indicate the occurrence of allergic reaction in the patient. The serum IgE titer corresponds to the total amount of allergen-specific IgE. Though it is easy to determine the total amount of IgE when any allergen is tested, the IgE titer may be reduced in some patients, such as, those with non-atopic bronchitis.

The number of eosinophils and eosinophil cationic protein (ECP) level are diagnostic items for delayed-type reaction following Type I allergy and allergic inflammatory reaction. The number of eosinophils is considered to reflect the progress of allergic symptoms. ECP, a protein contained in eosinophil granules, is also strongly activated in patients with an asthma attack. Indeed these diagnostic items identify allergy symptoms, but they are limited in their scope as diagnostic indicators.

Therefore, a marker (indicator) for an allergic disease that is not only less invasive to patients but also capable of readily providing information necessary for diagnosis would be useful. Since such markers are thought to be deeply involved in disease onset, they may be an important target in the control of allergic symptoms as well as in diagnosis.

### SUMMARY OF THE INVENTION

An objective of the present invention is to provide an indicator gene for testing an allergic disease. Another objective of the invention is to provide a method of testing for an allergic disease and a method of screening for a therapeutic agent for an allergic disease, both using the expression of the gene as an indicator.

Based on a previously established technique, the “fluorescent differential display method (Fluorescent DD method)” (T. Ito et al. 1994, FEBS Lett. 351: 231-236), the present inventors developed a new DD system capable of analyzing T-cell RNA samples prepared from multiple human blood samples. Using Fluorescent DD method, the present inventors successfully isolated genes whose expression levels change in peripheral blood cells from patients with pollinosis. The genes that were isolated (along with the patent applications filed) are listed below:

Pollinosis-associated gene 373 (WO 00/65046),

Pollinosis-associated gene 419 (WO 00/65045),

Pollinosis-associated gene 513 (WO 00/65049),

Pollinosis-associated gene 581 (WO 00/65048),

Pollinosis-associated gene 795 (WO 00/65050),

Pollinosis-associated gene 627 (WO 00/65051),

Pollinosis-associated gene 441 (WO 00/73435),

Pollinosis-associated gene 465 (WO 00/73439), and

Pollinosis-associated gene 787 (WO 00/73440).

Similarly, genes whose expression levels in peripheral blood differ between patients of allergic diseases and healthy subjects were searched using differential display method. As a result, gene B1153 whose expression level significantly rose in the patient group was discovered and filed as a patent application (WO 02/50269).

The present inventors used the Fluorescent DD method to isolate genes whose expression level is altered in an allergic disease-specific manner. Specifically, first, the present inventors collected blood from normal healthy subjects and patients with allergic diseases (atopic dermatitis and allergic asthma), isolated T cells from the blood, and via the Fluorescent DD method screened for genes whose expression levels differ between the normal healthy subject group and the patient group. As a result, the present inventors succeeded in isolating a gene, “B1799,” that showed significantly higher expression levels in the patient group. The isolated gene was the same as KIAA0603 (GenBank Accession No. AB011175). Genomic sequence information and expression data are available for the mouse homologue of KIAA0603 (GenBank Accession No. AB011175; Genomics 2002 February; 79(2):154-61 Candidate genes required for embryonic development: a comparative analysis of distal mouse chromosome 14 and human chromosome 13q22. Kurihara L J, Semenova E, Miller W, Ingram R S, Guan X J, Tilghman S M. Howard Hughes Medical Institute and Department of Molecular Biology, Princeton University, Princeton, N.J. 08544, USA). According to the report, sequences adjacent to the gene encoding Endothelin B receptor were determined, and the mouse homologue was shown as one of the genes comprised in this adjacent region. Furthermore, since deletion of this region in mice led to embryonic death, these genes were suggested to be related to this incident. No information relating to immunity and allergy is included in the report. The isolated human B1799 gene encodes a protein consisting of 1299 amino acids. This protein has the characteristic of a GTPase activating protein, which indicated that it is involved in cell differentiation and cell cycle control. The expression level of the B1799 gene was higher in atopic dermatitis patients compared to normal healthy subjects, especially higher in patients with moderate symptom. Analysis of the expression of the gene in various types of leukocytes revealed that the expression was high particularly in T cells, more particularly in memory T cells (cells with the phenotypes of CD4⁺ and CD45RO⁺). Furthermore, the present inventors discovered from the result of in vitro stimulation test that expression of B1799 mRNA is induced during the activated cell death of T cells. Specifically, B1799 mRNA expression was induced under conditions that activated the T cells by stimulus via T cell receptors or stimulus such as calcium ionophores (ionomycins). This relation of B1799 gene expression with the activation of the T cells verifies the involvement of the gene in allergic diseases. Thus, the inventors found that testing for allergic diseases and screening of candidate compounds serving as therapeutic agents for allergic diseases are possible by using the expression level of the gene as an indicator, and completed this invention.

The present invention relates to a method of testing for allergic diseases and a method of screening for therapeutic agents for allergic diseases based on the expression level of an indicator gene, the B1799 gene, which shows a high expression in allergic disease patients. Specifically, the present invention relates to the following testing methods for allergic diseases, kits therefor, and methods of screening for therapeutic agents for allergic diseases:

(1) A method of testing for an allergic disease, the method comprising the steps of:

(a) measuring the expression level of an indicator gene in a biological sample from a test subject;

(b) comparing the expression level with that of the indicator gene in a biological sample from a healthy subject; and

(c) judging the test subject to have an allergic disease when the expression level of the indicator gene in the biological sample from the test subject is found to be significantly elevated, wherein the indicator gene is B1799 gene.

(2) The testing method according to (1), wherein the allergic disease is atopic dermatitis.

(3) The testing method according to (1), wherein the gene expression level is measured via cDNA PCR.

(4) The testing method according to (1), wherein the gene expression level is measured by detecting a protein encoded by the gene.

(5) The testing method according to (1), wherein the biological sample contains peripheral blood T cells.

(6) A reagent for diagnosis of an allergic disease, said reagent comprising a polynucleotide that comprises at least 15 continuous nucleotide sequence of B1799 gene or a complementary sequence thereof;

(7) A reagent for testing for an allergic disease, said reagent comprising an antibody that binds to a polypeptide consisting of the amino acid sequence encoded by B1799 gene;

(8) A method for screening a therapeutic agent for an allergic disease, wherein said method comprises the steps of:

(a) contacting a candidate compound with a cell expressing an indicator gene;

(b) measuring the expression level of said indicator gene; and

(c) selecting a compound which decreases the expression level of the indicator gene as compared to a control where said candidate compound has not been contacted,

wherein the indicator gene is B1799 gene.

(9) The method according to (8), wherein the cell is T cell.

(10) A method of screening for a therapeutic agent for an allergic disease, the method comprising the steps of:

(a) administering a candidate compound to a test animal;

(b) measuring the expression level of an indicator gene in leukocytes of the test animal; and

(c) selecting a compound which decreases the expression level of the indicator gene compared to a control where the candidate compound has not been administered,

wherein the indicator gene is B1799 gene.

(11) A method of screening for a therapeutic agent for an allergic disease, the method comprising the steps of:

(a) contacting a candidate compound with cells containing a reporter gene linked under the control of transcriptional regulatory region of an indicator gene;

(b) measuring the expression level of the reporter gene; and

(c) selecting a compound which decreases the expression level of the reporter gene compared to a control where the candidate compound has not been contacted,

wherein the indicator gene is B1799 gene.

(12) A method of screening for a therapeutic agent for an allergic disease, the method comprising the steps of:

(a) contacting a candidate compound with a protein encoded by an indicator gene or a gene functionally equivalent thereto;

(b) measuring the activity of the protein; and

(c) selecting a compound which decreases the activity of the protein compared to a control where the candidate compound has not been contacted,

wherein the indicator gene is B1799 gene.

(13) A therapeutic agent for an allergic disease comprising as the main ingredient a compound obtainable by the screening method according to any one of (8), (10), (11), and (12).

(14) A therapeutic agent for an allergic disease, which comprises as a main ingredient an antisense DNA that contains a sequence complementary to a sequence comprising at least 15 continuous nucleotides of the sense strand sequence of an indicator gene, wherein the indicator gene is B1799 gene.

(15) A therapeutic agent for an allergic disease, which comprises as a main ingredient an antibody which binds to a protein encoded by an indicator gene, wherein the indicator gene is B1799 gene.

(16) An animal model of allergic disease, wherein said animal is a transgenic nonhuman vertebrate, in which the expression level of an indicator gene, or a gene functionally equivalent thereto, has been increased in T cells, wherein the indicator gene is B1799 gene.

(17) A kit for screening for a therapeutic agent for an allergic disease, the kit comprising a polynucleotide comprising at least 15 continuous nucleotide sequence of an indicator gene or its complementary sequence, wherein the indicator gene is B1799 gene.

(18) A kit for screening for a therapeutic agent for an allergic disease, the kit comprising an antibody which binds to a polypeptide consisting an amino acid sequence encoded by an indicator gene and cells expressing the indicator gene, wherein the indicator gene is B1799 gene.

The present invention also relates to a method of treating an allergic disease, which comprises the step of administering a compound of any one of the following (A) to (C). In addition, the present invention relates to the use of a compound of any one of the following (A) to (C) for producing a therapeutic agent for an allergic disease:

(A) a compound obtainable by the screening method according to any one of (8), (10), (11), and (12) above;

(B) an antisense DNA having a sequence complementary to a sequence comprising at least 15 continuous nucleotides of the sense strand sequence of B1799 gene; and

(C) an antibody which binds to a protein encoded by B1799 gene.

Furthermore, the present invention relates to a method of producing an allergic disease animal model, the method comprising the step of increasing the expression level of B1799 gene or a gene functionally equivalent thereto in T cells in a nonhuman vertebrate. In addition, the present invention relates to the use of a transgenic nonhuman vertebrate, in which the expression level of B1799 gene or a gene functionally equivalent thereto has been increased, as an allergic disease animal model.

### BRIEF DESCRIPTION OF THE DRAWINGS

FIG. 1 shows the amount of expression of B1799 in atopic dermatitis patient samples.

FIG. 2 shows the amount of expression of B1799 in leukocytes in peripheral blood.

FIG. 3 shows the amount of expression of B1799 in T cell subgroups derived from peripheral blood.

FIG. 4 shows the (a) the change in the rate of dead cells during T cell activated cell death, and (b) the change in the expression of B1799 during T cell activated cell death.

FIG. 5 shows the change in the expression of B1799 due to T cell stimulation.

FIG. 6 shows the structure of B1799.

FIG. 7 shows the predicted tertiary structure model of B1799 protein. Arg343 of Gyp1p and Arg973 of B1799 are shown by arrows.

### DETAILED DESCRIPTION OF THE INVENTION

The present invention relates to a method of testing for allergic diseases using the expression level of B1799 gene as an indicator in leukocytes of a test subject. According to the present invention, the B1799 gene was demonstrated to show a higher expression level in a patient group with allergic diseases as compared to the group of healthy subjects. Therefore, allergic diseases can be tested using the expression level of the B1799 gene as an indicator. Herein, the B1799 gene is referred to as “the indicator gene.” The term “allergic disease” used herein is a general term for diseases involving allergic reactions. More specifically, this term is defined as a disease for which an allergen is identified, a strong correlation between exposure to the allergen and the onset of the pathological change is demonstrated, and the pathological change has been proven to have an immunological mechanism. Herein, an immunological mechanism means that leukocytes show an immune response to allergen stimulation. Examples of allergens are mite antigens, pollen antigens, etc.

Representative allergic diseases include atopic dermatitis, allergic rhinitis, pollinosis, bronchial asthma and insect allergy. Allergic diathesis is a genetic factor that is inherited from allergic parents to children. Familial allergic diseases are also called atopic diseases, and their causative factor that can be inherited is atopic diathesis. Among allergic diseases, asthma is a general term for diseases accompanied with respiratory organ symptoms.

As used herein, the term “B1799 gene” refers to human B1799 gene identified in Examples and a gene located in the same locus or a gene located in a homologous locus in another organism. For example, in the present invention, the B1799 gene includes an arbitrary allele in the same locus as or homologous locus to that of the human B1799 gene identified in Examples. The nucleotide sequence of the transcription product of the human B1799 gene identified in Examples and the amino acid sequence of a protein encoded by the gene is shown in SEQ ID Nos: 1 and 2, respectively. Specifically, in the present invention, the B1799 gene includes the human B1799 gene (SEQ ID NO:1) identified in Examples, its polymorphic variants (including SNPs), mutants, splicing variants, and homologues from other organisms. Since the expressional control for these genes are presumed to be substantially identical or similar to that of the human B1799 gene shown in SEQ ID NO:1, tests for allergic diseases according to the present invention can be carried out by detecting their expression.

Specifically, in the present invention, the B1799 gene is substantially identical to an endogenous gene that comprises the following nucleic acid. The endogenous gene refers to an artificially unmodified gene contained in an organism in nature, and a gene that comprises the same nucleotide sequence is substantially identical to the endogenous gene. Furthermore, herein the term “gene” refers to a transcription product or nucleic acid (e.g., DNA, RNA, etc.) encoding the same.

(a) A nucleic acid that comprises a nucleotide sequence selected from the nucleotide sequence of SEQ ID NO:1.

(b) A nucleic acid comprising a nucleotide sequence of a coding sequence of SEQ ID NO:1, which contains one or more nucleotide substitutions, deletions and/or insertions.

(c) A nucleic acid encoding at least 15 continuous amino acids of the amino acid sequence of SEQ ID NO:2.

(d) A nucleic acid which encodes a protein comprising the amino acid sequence of SEQ ID NO:2 containing one or more amino acid substitutions, deletions and/or insertions.

(e) A nucleic acid which hybridizes under a stringent condition to a nucleic acid containing at least 50 continuous nucleotides of SEQ ID NO:1 but no nucleotide sequence derived from any sequence other than SEQ ID NO:1.

The nucleic acid according to (a) includes those containing preferably at least 40, more preferably 60, 100, 200, 500, and 1,000 or more continuous nucleotides of the nucleotide sequence of SEQ ID NO:1 (more preferably the coding region of SEQ ID NO:1; nt 348 to nt 4244), and most preferably its 5922 nucleotides (i.e., the whole length). Such nucleic acids include polymorphic variants, mutants, and splicing variants of the gene primarily within the same species. Examples of the nucleic acid according to (a) are those containing the nucleotide sequences of at least 30, more preferably 35, 40, 45, and 50 or more continuous nucleotides of the nucleotide sequence of SEQ ID NO:1. Nucleotide sequence is preferably selected from the coding region of SEQ ID NO:1 (nt 348 to nt 4244), and any desirable numbers of nucleotide sequences may be selected. In the case of selecting a plurality of nucleotide sequences, it is desirable to select nucleic acids comprising the continuous nucleotide sequences at 2 or more, preferably 3, 4, and 5 or more different sites so that the nucleic acids do not overlap.

The nucleic acid according to (b) includes those containing the nucleotide sequences of the coding region of SEQ ID NO:1 (nt 348 to nt 4244 in SEQ ID NO:1) in which preferably 15% or less, more preferably 10%, 8%, 5%, and 1% or less of the total nucleotides is substituted, deleted, and/or inserted. Such nucleic acids may include counterpart genes of other close relative species, polymorphic variants, mutants, and splicing variants thereof. Such nucleic acids are those containing nucleotide sequences having the sequence identity of preferably 85% or more, more preferably 90%, 92%, 95%, and 99% or more to the coding region set forth in SEQ ID NO: 1.

Nucleic acid or amino acid sequence identity can be determined using, for example, a BLAST program (Altschul, S. F. et al., 1990, J. Mol. Biol. 215: 403-410). More specifically, nucleotide sequence identity is determined using the blastn program, while amino acid sequence identity using blastp program, and sequence identity search is conducted, for example, using default parameters with all filters containing Low complexity off at the BLAST website of the National Center for Biotechnology Information (NCBI) (Altschul, S. F. et al. (1993) Nature Genet. 3: 266-272; Madden, T. L. et al. (1996) Meth. Enzymol. 266: 131-141; Altschul, S. F. et al. (1997) Nucleic Acids Res. 25: 3389-3402; Zhang, J. and Madden, T. L. (1997) Genome Res. 7: 649-656). For example, using the blast2sequences program (Tatiana, A. et al. (1999) FEMS Microbiol Lett. 174: 247-250) for comparing two sequences, the two sequences can be aligned to determine the sequence identity. In this case, gaps are similarly treated as mismatches to calculate the identity value for the entire coding region of SEQ ID NO:1.

The nucleic acid according to (c) includes those encoding preferably at least 20, more preferably 30, 50, 100, 300, and 500 or more continuous amino acids of the amino acid sequence of SEQ ID NO:2, and most preferably its 1299 amino acids (i.e., the whole length). Such nucleic acids include, similarly as in the above-described (a), polymorphic variants, mutants, and splicing variants of genes primarily within the same species of organism. Furthermore, the nucleic acid according to (c) includes those containing partial nucleotide sequences encoding the amino acid sequence of at least 8, more preferably 10, 15, 20, and 25 or more continuous amino acids of SEQ ID NO:2 at 2 or more, preferably 3 or more, more preferably 4 and 5 or more sites so that each nucleic acids do not overlap.

The nucleic acid according to (d) includes those encoding the amino acid sequence of SEQ ID NO:2 in which preferably 15% or less, more preferably 10%, 8%, 5%, and 1% or less of the whole number of its amino acid residues are substituted, deleted, and/or inserted. These nucleic acids may contain untranslated sequences. Such nucleic acids include a counterpart gene of other close relative species, polymorphic variants, mutants, and splicing variants. Such nucleic acids are those encoding proteins comprising amino acid sequences having the sequence identity of preferably 85% or more, more preferably 90%, 92%, 95%, and 99% or more to the amino acid sequence of SEQ ID NO:2. Treating gaps similar to mismatches, the identity value relative to the whole amino acid sequence of SEQ ID NO:2 is calculated. Amino acid sequence identity can be determined according to the above-described method.

The nucleic acid according to (e) includes those hybridizing to nucleic acids comprising preferably at least 80, more preferably 100, 120, and 200 or more continuous nucleotides of SEQ ID NO:1 (more preferably its coding region; nt 348 to nt 4244), and substantially containing no other nucleotide sequence than that of SEQ ID NO:1 (more preferably its coding region; nt 348 to nt 4244) under the stringent condition. Such nucleic acids may be those hybridizing under the stringent condition to probes that have been prepared, for example, using the nucleic acid containing the nucleotide sequence of SEQ ID NO:1 as a template. Probes of 50 to several hundreds of nucleotides long can be synthesized, for example, by the DNA synthesis method, or by the random prime method, nick translation method and PCR method.

The stringent condition for the nucleic acid according to (e) is that hybridization is carried out in a hybridization solution preferably containing NaCl in the range of about 0.5 to about 0.9 M at 60° C., preferably at 62° C., more preferably at 65° C., and then washing is performed at the same temperature as for hybridization in 1×SSC, preferably in 0.5×SSC, more preferably in 0.2×SSC, still more preferably in 0.1×SSC for 1 h. In this case, the temperature conditions of hybridization and washing that greatly influence the stringency can be adjusted according to the melting temperature (Tm) of the probe, which depends on the ratio of constituent nucleotides of the probe to the base pairs to which the probe hybridizes, the length of the probe, and the composition (concentrations of salts and formamide) of a hybridization solution. Considering these conditions, those skilled in the art can empirically set up the appropriate condition to confer the equivalent stringency. As a hybridization solution, for example, 4×SSC, preferably ExpressHyb™ Hybridization Solution (Clontech) etc. may be used.

The B1799 gene preferably encodes a GTPase activating protein (GAP). GAP is a protein binding to the GTP binding protein. The GTPase activity of the GTP binding protein is enhanced by the interaction with GAP (Scheffzek, K. et al., Trends Biochem. Sci., 1998, 23(7): 257-262). The GTP binding protein that plays an important role in signal transduction, is activated when GTP binds to it and inactivated when the bound GTP is converted to GDP. The GTP binding protein itself has GTPase activity, and GAP enhances its GTPase activity. GAP controls signal transduction by assisting conversion of the GTP binding protein to the inactivated form. Furthermore, the B1799 gene of the present invention preferably encodes a protein having a phosphotyrosine interaction domain (PID) that is involved in the binding between proteins at the phosphotyrosine binding domain and a TBC domain. The PID domain is conserved in proteins interacting with phophotyrosine, and is found in a group of proteins involved in various biological processes comprising signal transduction mediated by cell surface receptors or occurring during protein transport (Bork, P. and Margolis, B., 1995, Cell 80(5): 693-694). The TBC domain was discovered in Tbc1 (Richardson, P. M. and Zon, L. I., 1995, Oncogene 11(6): 1139-1148), and is commonly seen in oncogene tre-2 and yeast cell cycle regulating factor BUB2 and cdc16. The TBC domain exists in Gyp6 and Gyp7, and is considered to be a domain widely distributed in GAPs of Rab-like GTPases (Neuwald, A. F., 1997, Trends Biochem. Sci. 22(7): 243-244).

In a preferred embodiment of the present invention, the B1799 gene is a nucleic acid encoding a transcription product, which is transcribed from a promoter regulating the transcription of a genomic DNA that encodes mRNA comprising the nucleic acid of SEQ ID NO: 1 in a native human cell or from the counterpart of the promoter in other species.

Specifically, the method of testing for allergic diseases according to the present invention includes the steps of: (a) measuring the expression level of the indicator gene (i.e., B1799 gene) in a biological sample from a test subject; (b) comparing the expression level with that in a corresponding sample from a healthy subject; and (c) judging the test subject as being affected with an allergic disease if the expression level of the indicator gene in the biological sample of the test subject is enhanced compared to that in the healthy subject. Since the B1799 gene can be used as an indicator in the present invention, the B1799 gene is herein also called simply an indicator gene. As described above, the B1799 gene of the present invention includes, beside the human gene, homologues of other species. Therefore, unless otherwise stated, B1799 gene of species other than human means endogenous homologues in those species. If the expression level of the B1799 gene in a test subject is higher than that in a healthy subject, the test subject is judged to have an allergic disease. Alternatively, a standard value may be set in advance based on the expression level of the B1799 gene in a healthy subject, and the expression level in a test subject can be compared to the standard value. Methods to set a standard value and acceptable range based on the measured value of the indicator gene are well known in the art. For example, a range of ±2 S.D. can be used as the acceptable range. If the expression level of the indicator gene in the test subject is within the acceptable range, the test subject is suggested to be normal with respect to allergic diseases; and if the expression level is over the range, the subject is judged to have an allergic disease.

The present testing method can be conducted by using not only the B1799 gene alone but its combination with other genes that serve as an indicator for allergic diseases. The accuracy of the test can be improved by detecting the expression of multiple genes in combination. Generally, allergic disease patients form a heterogeneous population, and thus, a more accurate diagnosis is enabled by using multiple genes as indicators.

In this invention, expression of the indicator gene includes both transcription and translation. That is, the expression level of the indicator gene can be the level of mRNA or protein corresponding to the gene. Therefore, the method of testing for allergic diseases of the present invention may be performed based on comparing the level of mRNA corresponding to the indicator gene, or the level of the protein encoded by the gene. The measurement of the mRNA level can be carried out according to known genetic analysis methods. Specifically, one can use, for example, a hybridization technique using the nucleic acids that hybridize to the indicator gene as probes, or a gene amplification technique using DNAs that hybridize to that gene as primers.

The probes or primers used for the testing of the present invention can be designed based on the nucleotide sequence of the indicator gene. For example, the nucleotide sequence of the human B1799 gene identified in Example and the amino acid sequence encoded by the gene are described in SEQ ID NOs: 1 and 2, respectively. In general, genes of higher animals are often accompanied by polymorphism. Many molecules produce isoforms comprising different amino acid sequences from each other during the splicing process. Any genes can be used as the indicator gene of the present invention, even though they differ from the above-mentioned gene in the nucleotide sequence due to polymorphism, alternative splicing, and such.

As a primer or probe can be used a polynucleotide comprising at least 15, preferably at least 20, more preferably at least 30, and much more preferably at least 35 continuous nucleotides of the nucleotide sequence of the B1799 gene or its complementary sequence. Herein, the term “complementary sequence” refers to the nucleotide sequence of one strand of a double stranded polynucleotide, which is composed of A:T (or A:U) and G:C base pairs, to the other strand. Furthermore, such polynucleotides may comprise nucleotide sequences other than a part of the nucleotide sequence of B1799 or its complementary sequence. In addition, polynucleotides that are at least 70%, preferably 80% or more, more preferably 90% or more, and much more preferably 95% or more identical to the nucleotide sequence of at least 20, preferably 30, more preferably 35, and much more preferably 40 continuous nucleotides of the B1799 gene or its complementary sequence can be used as reagents for testing allergic diseases of the present invention. The degree of homology between nucleotide sequences can be determined by an algorithm, such as BLAST. The detection of the indicator gene is enabled by specific hybridization of the above-mentioned polynucleotides to the gene. A specific hybridization can be confirmed if there is no significant cross-hybridization with DNA and/or RNA encoding other genes under the above-mentioned stringent conditions.

Such polynucleotides are useful as probes to detect the indicator gene, and as primers to amplify the indicator gene. When used as a primer, those polynucleotides have a chain length of usually 15 bp to 100 bp, preferably 15 bp to 35 bp. When used as a probe, DNAs comprising the whole sequence of the indicator gene (or its complementary strand), or its partial sequence that contains at least 15-bp, are used. When used as a primer, its 3′ region must be complementary to the indicator gene, while the 5′region can be linked to a restriction enzyme-recognition sequence or tag.

“Polynucleotides” as used in the present invention may be either DNA or RNA. These polynucleotides may be either synthetic or naturally-occurring. Also, DNA used as a probe for hybridization is usually labeled. Examples of labeling methods are described below. Herein, the term “oligonucleotide” refers to polynucleotides with relatively low degree of polymerization. A chain length of an oligonucleotide is, for example, within 100 nucleotides. Oligonucleotides are included in polynucleotides. The labeling methods are as follows:

nick translation labeling using DNA polymerase I;

end labeling using polynucleotide kinase;

fill-in end labeling using Klenow fragment (Berger S L, Kimmel A R. (1987) Guide to Molecular Cloning Techniques, Methods in Enzymology, Academic Press; Hames B D, Higgins S J (1985) Genes Probes: A Practical Approach. TRL Press; Sambrook J, Fritsch E F, Maniatis T. (1989) Molecular Cloning: a Laboratory Manual, 2nd Edn. Cold Spring Harbor Laboratory Press);

transcription labeling using RNA polymerase (Melton D A, Krieg P A, Rebagkiati M R, Maniatis T, Zinn K, Green M R. (1984) Nucleic Acid Res., 12, 7035-7056); and

non-radioisotopic labeling of DNA by incorporating modified nucleotides (Kricka L J. (1992) Nonisotopic DNA Probing Techniques. Academic Press).

For testing for allergic diseases using hybridization techniques, for example, Northern hybridization, dot blot hybridization, or DNA microarray technique may be used. Furthermore, gene amplification techniques, such as RT-PCR method may be used. In RT-PCR, RNA is extracted from a biological sample, cDNA is prepared by its reverse transcription, and PCR is conducted using the obtained cDNA as the template to determine the gene expression level. By using the PCR amplification monitoring method during the gene amplification step in RT-PCR, one can achieve more quantitative analysis for the gene expression of the present invention.

In the PCR gene amplification-monitoring method, the detection target (DNA or reverse transcript of RNA) is hybridized to probes that are dual-labeled at both ends with different fluorescent dyes whose fluorescence cancels each other out. When the PCR proceeds and Taq polymerase degrades the probe as a result of its 5′-3′ exonuclease activity, the two fluorescent dyes become distant from each other and the fluorescence is detected. The fluorescence is detected in real time. By simultaneously measuring a standard sample in which the copy number of the target is known, it is possible to determine the copy number of the target in the subject sample with the cycle number where PCR amplification is linear (Holland P. M. et al., 1991, Proc. Natl. Acad. Sci. USA 88: 7276-7280; Livak K. J. et al., 1995, PCR Methods and Applications 4(6): 357-362; Heid C. A. et al., 1996, Genome Research 6: 986-994; Gibson E. M. U. et al., 1996, Genome Research 6: 995-1001). For the PCR amplification-monitoring method, for example, ABT PRISM7700 (PE Biosystems) may be used.

The method of testing for allergic diseases of the present invention can be also carried out by detecting a protein encoded by the indicator gene. Hereinafter, a protein encoded by the indicator gene is described as an indicator protein. For example, for such test methods Western blotting, immunoprecipitation method, and ELISA method may be employed using an antibody that binds to the indicator protein.

Antibodies that bind to the indicator protein used in the detection may be produced by techniques well known to those skilled in the art. Antibodies used in the present invention may be polyclonal or monoclonal antibodies (Milstein C. et al., 1983, Nature 305 (5934): 537-40). For example, polyclonal antibodies against the indicator protein may be produced by collecting blood from mammals sensitized with the antigen, and separating the serum from this blood using known methods. As polyclonal antibodies, serum containing polyclonal antibodies may be used. If desired, a fraction containing polyclonal antibody can be further isolated from this serum. Alternatively, monoclonal antibodies may be obtained by isolating immune cells from mammals sensitized with the antigen, fusing these cells with myeloma cells, and such, cloning the obtained hybridomas, and collecting the antibodies from the culture of the hybridomas.

For detecting an indicator protein, these antibodies may be appropriately labeled. Alternatively, instead of labeling the antibody, a substance that specifically binds to the antibody, for example, protein A or protein G, may be labeled to indirectly detect the indicator protein. Specifically, one example of a detection method is ELISA.

A protein or its partial peptide used as an antigen may be obtained, for example, by inserting a gene encoding the protein or its portion into an expression vector, introducing the vector into an appropriate host cell to produce a transformant, culturing the transformant to express the recombinant protein, and purifying the expressed recombinant protein from the culture or the culture supernatant. Alternatively, oligopeptides consisting of a partial amino acid sequence of the protein can be chemically synthesized to be used as the immunogen.

Furthermore, the method of testing for allergic diseases of the present invention can be conducted using the activity of indicator protein in leukocytes as an indicator. The activity of indicator protein refers to the biological activity possessed by the protein. More specifically, activity such as GAP activity, i.e. activation of the GTP binding protein, can be mentioned as the activity of the indicator protein of the present invention. Alternatively, its interaction with the GTP binding protein can be used as the index of the activity. Detection of the activity of the indicator protein can be preformed based on conventional methods. More specifically, the GAP activity can be determined by measuring the GTPase activity of a specific GTP binding protein on which this protein acts. For example, Gyp6 protein specifically acts on Ypt6 protein, a GTP binding protein, and enhances its GTPase activity. Therefore, by detecting the increase in GTPase activity of Ypt6 protein, the activity of Gyp6 protein can be measured (Strom M. et al., 1993, Nature 361(6414): 736-739). Thus, by measuring the activity of a specific GTP binding protein on which the indicator protein B1799 of the present invention acts, the activity of the indicator protein can be measured.

Normally, in the testing method of this invention, a biological sample from a subject is used as a test sample. A biological sample such as peripheral blood T cells can be used. Leukocytes used for the test may be purified or partially purified. Preferably, leukocytes prepared from peripheral blood, more preferably T-cells, of subjects are used as the test sample. T-cells can be prepared from peripheral blood by known methods. Specifically, for example, heparinized blood is collected, diluted, fractionated by centrifugation using Ficoll to separate PBMC. The separated PBMC may be used as it is as the sample for the test for allergic diseases of the present invention. PBMC contains lymphocytes and monocytes. As shown in Examples, among these blood cells, the indicator gene is highly expressed in T cells. The expression level of the indicator gene can be measured without great influence of cells other than T cells. Direct analysis of not a purified T-cell fraction but a lymphocyte fraction as a test sample enables a convenient bed-side test. Alternatively, T cells can be isolated by allowing them to be specifically adsorbed by microbeads to which the anti-CD3 antibody has been immobilized.

The intracellular mRNA of the indicator gene or the indicator protein can be measured in disintegrated blood cells. It is also possible to measure the indicator protein in blood. For the preparation of T cell lysate and extraction of mRNA, kits such as an RNeasy Mini™ (Qiagen) and ISOGEN™ (Nippon Gene) on the market may be conveniently used.

The measured value of expression level of the indicator gene in test biological samples can be corrected by known methods. The changes in the gene expression level in the biological samples can be compared using the corrected values. The measured value of the expression level of the genes that are to be used as indicators in the present invention may be corrected based on the measured value of the expression level of the genes (such as housekeeping genes) that are expressed in the biological samples and do not largely fluctuate in their expression levels regardless of the condition of the cell. Examples of such genes are β-actin gene and glyceraldehyde 3-phosphate dehydrogenase (GAPDH) gene.

According to the test method of the present invention, the expression level of the indicator gene in leukocytes from a test subject and the expression level in the corresponding leukocytes from a healthy subject are measured. It is examined whether the expression level in the leukocytes of the test subject is increased compared to that in the healthy subject. The increase in the expression level of the indicator gene in the leukocytes from the test subject is correlated with allergic diseases.

The testing for an allergic disease according to the present invention includes, for example, those as described below. Even a patient who cannot be judged as having an allergic disease by the ordinary testing in spite of showing allergic disease-like symptoms can be easily judged as an allergic disease patient by the testing of this invention. More specifically, the elevation of the indicator gene expression in patients with symptoms suspect of an allergic disease indicates that the symptoms would probably be those of an allergic disease. Allergic disease-like symptoms are exemplified by dermatitis (itching, flare), rhinitis (nasal congestion, running nose, sneeze), and asthma (stridor, dyspnea). The method of testing for allergic diseases according to the present invention includes tests for determining whether such allergic disease-like symptomsare caused by allergic reaction. Although these symptoms are also observed in xeroderma, cold syndrome (coryza), bronchitis, and such, it is possible to determine whether these symptoms are caused by allergic reaction or not, according to the test method of the present invention. In addition, the method of testing for allergic diseases of the present invention includes tests to determine whether a subject has allergic diathesis or not. Specifically, the test is conducted for a subject without noticeable symptom, and the subject is determined to have allergic diathesis if an increase in the expression level of the indicator gene is detected. For treatment of an allergic disease-like symptom, it is a very important process to determine whether allergic reaction is the cause of the symptom or not. The testing method of this invention can provide extremely important information for identifying the cause of diseases. Alternatively, even without referring to information of symptoms, allergic disease patients can be screened by testing samples and by screening samples that highly express the indicator gene.

In addition, the testing method is useful to judge whether allergic symptoms are getting ameliorated or not. The expression level of the indicator gene of this invention increases in T cells which is contained in PBMC of patients with an allergic disease. T cells play a central role in immune response. Therefore, the decreased expression level of the indicator gene in patients diagnosed as having an allergic disease indicates amelioration of allergic symptoms, and the increased expression level indicates that the allergic symptoms would be in progress.

Furthermore, the expression of the indicator gene B1799 has been revealed to be high in T cells among leukocytes, especially in T cells having the phenotypes of CD4⁺ and CD45RO⁺ (i.e., memory T cells). Therefore, the indicator gene can be used as a marker for these T cells. Moreover, the expression of the indicator gene is significantly induced in activated cell death of T cells. In addition, the expression is also induced by activation stimuli by a stimulant such as ionomycin. Thus, since the expression of the indicator gene reflects the state of T cell activation, the activation level of T cells may be determined by measuring the expression of the indicator gene in T cells.

The present invention also relates to the use of a transgenic nonhuman animal, in which the expression level of the indicator gene B1799 or a gene functionally equivalent thereto is elevated in T cells, as an allergic disease model animal. The allergic disease model animal is useful in clarifying changes in vivo in allergy. Furthermore, the allergic disease model animal of this invention is useful in the assessment of therapeutic agents for an allergic disease.

The present invention demonstrated the elevated expression level of the indicator gene in T cells. Therefore, animals in which the expression level of the B1799 gene or a gene functionally equivalent thereto is artificially elevated in T cells can be utilized as the allergic disease model animal. Herein, transgenic animals refer to animals genetically modified by insertion, substitution, and/or deletion of one or more nucleotides in their genomic DNAs. This allergic disease model animal is useful in clarifying changes in vivo in allergy. Furthermore, the allergic disease model animal of this invention is useful in assessing and screening for therapeutic agents for an allergic disease. In this case, the increased expression level in T cells includes the increased expression level of the indicator gene in the whole blood cells. That is, the expression level elevation of the indicator gene includes that in not only T cells but also blood cells as a whole or the whole body.

Genes functionally equivalent to the B1799 gene used in the present invention refer to those encoding proteins having the activity equivalent to that of the protein (indicator protein) encoded by the indicator gene B1799. Such genes include artificial variants of the human B1799 gene or its counterparts from other organisms. For example, in the case of inserting a gene into an expression vector, deletion of nucleotides corresponding to several amino acid residues at the N- or C-terminus, and addition of nucleotides corresponding to a tag peptide sequence or other amino acid sequence derived from the expression vector are frequently performed. The resulting proteins are different in the amino acid sequence from the endogenous protein in natural cells, but their activities are substantially equivalent to that of the endogenous protein. Herein, genes encoding such proteins are referred to as genes functionally equivalent to the endogenous gene.

One example of the activities of the indicator protein used in this invention is the GAP activity. For example, a gene encoding the human B1799 protein of SEQ ID NO:2 in which one or more amino acids are deleted, inserted, and/or substituted and which maintains the GAP activity, can be used to prepare the transgenic model animal of this invention. Such modified proteins should have a homology of, for example, 90% or more, preferably 95% or more and, still more preferably 99% or more to the amino acid sequence of the human B1799 protein. Furthermore, genes which comprises nucleotide sequences hybridizing to the sequence of SEQ ID NO:1 under stringent conditions, and encode proteins having the GAP activity, can also be used as genes functionally equivalent to the B1799 gene.

The model animal of this invention can be used as a model for an allergic disease, for example, in a method comprising the steps of: (a) detecting a phenotype of the model animal, and (b) correlating the difference of the phenotype of said model animal from the corresponding phenotype of a control animal, whose expression level of the indicator gene in T cells is lower compared to that of said model animal, with an allergic disease. Examples of the control animals include non-transgenic animal having the same genetic background as that of said model animal and a transgenic animal in which an empty vector has been introduced. The phenotype may be any desired phenotypes including allergic symptoms such as dermatitis, rhinitis, and asthma, or the activation of immunocytes or changes in gene expression.

In the present invention, it is highly significant to assess roles of the indicator gene B1799 and effects of drugs targeting this gene using transgenic animals in which the expression level of the indicator gene is elevated in T cells, as the allergic disease model animal. Furthermore, the allergic disease model animals according to the present invention are useful not only in screening for pharmaceutical agents for the treatment or prophylaxis of an allergic disease as described below but also in elucidating the mechanism of allergic diseases, and, furthermore, testing the safety of screened compounds. For example, if the allergic disease model animals according to this invention either develop clinical manifestations of atopic dermatitis or allergic asthma, or show changes in measured values associated with any allergic diseases, it is possible to construct a system for screening for compounds that can cure the allergic conditions.

Herein, the elevation of the expression level refers to any of the states that: a target gene introduced as an exogenous gene is allowed to be expressed; transcription of a gene inherent in the host and/or its translation into protein are/is increased; and, decomposition of the protein that is a translation product is suppressed. The gene expression level can be confirmed by, for example, quantitative PCR as described in Examples. Furthermore, the expression level or activity of the translation product protein can be confirmed by comparing it with that in a normal state.

A typical transgenic animal is an animal which has been transfected with a gene of interest and allowed to express the gene. In another type of transgenic animals, the half-life of mRNA may be extended by removing a sequence from the untranslated region (UTR) of mRNA, which renders RNA unstable. Furthermore, in another type of transgenic animals, a mutation is introduced into the coding region of the indicator gene to increase its activity or modify the amino acid sequence of the gene product protein so as to be hardly decomposed. Examples of mutation in the amino acid sequence are substitution, deletion, insertion, or addition of amino acid residues. In addition, mutation in the transcriptional regulatory region of the indicator gene also enables to enhance the expression of the gene.

Methods for obtaining transgenic animals targeting a specific gene are well-known in the art. That is, a transgenic animal can be obtained by a method where the gene and ovum are mixed and treated with calcium phosphate; a method where the gene is directly introduced into pronuclei of oocyte under a phase contrast microscope using a micropipette (microinjection method, U.S. Pat. No. 4,873,191); a method where the gene is introduced into embryonic stem cells (ES cells), etc. Furthermore, other methods include a method where ovum is infected with a gene-inserted retroviral vector and a sperm vector method where a gene is introduced into ovum mediated by sperm. The sperm vector method is a gene recombination technique for introducing an exogenous gene and performed by allowing an exogenous gene to adhere to a sperm or to be incorporated in a sperm by the electroporation method or such and fertilizing ovum with the sperm (M. Lavitranoet, et al., Cell, 57, 717, 1989).

Transgenic animals used as the allergic disease model animal of the present invention can be produced using all the vertebrates except for humans. More specifically, transgenic animals in which various genes have been introduced and their expression levels are modified are produced using vertebrates such as mice, rats, rabbits, miniature pigs, goats, sheep, monkeys, and cattle.

Furthermore, the present invention relates to methods of screening for therapeutic agents for allergic diseases. According to the present invention, the indicator gene shows a statistically significant increase in its expression level in the case of allergic diseases. Therefore, it is possible to obtain a therapeutic agent for an allergic disease by selecting a compound capable of reducing the expression level of this gene. Herein, compounds that reduce the expression level of a gene refer to those having inhibitory effects on any steps of the transcription or translation of the gene, stability of a transcript (mRNA) or a translation product (protein), and the expression of a gene function such as protein activity.

A method of screening for a therapeutic agent for allergic diseases of the present invention can be carried out either in vivo or in vitro. The in vivo screening method can be carried out, by the steps of:

(a) administering a candidate compound to a test animal;

(b) measuring the expression level of the indicator gene in leukocytes of the test animal; and

(c) selecting the compound that reduces the expression level of the indicator gene, compared to a control without administration of the compound.

As a test animal in the screening method of the present invention, any desirable animal that expresses the indicator gene of the present invention may be used. Specifically, such animals include, for example, mice, rats, guinea pigs, rabbits, cats, pigs, miniature pigs, goats, cattle, sheep, and monkeys. Preferably animals with elevated expression of the indicator gene are used. Furthermore, by using animals that exhibit allergic symptoms, the effect of candidate compounds on the symptoms can be evaluated.

The effect of a candidate drug compound can be detected by administering the candidate drug compound to a test animal and monitoring the action of the compound on the expression level of the indicator gene in leukocytes of the animal. The change in the expression level of the indicator gene in leukocytes of the test animal can be monitored by the same method as the above-mentioned test method of the present invention. Based on the detection result, candidate drug compounds for allergic diseases can be screened by selecting candidate drug compounds that reduce the expression level of the indicator gene.

More specifically, the screening of the present invention can be carried out by collecting leukocytes from a test animal to which a candidate compound has been given, and comparing the expression level of the indicator gene with that in corresponding leukocytes collected from an animal without administration of the candidate compound. Leukocytes used for the test may be purified or crude samples and include PBMC and purified T cells. Preferably, purified T-cells are used. Methods for collecting and preparing these biological samples are known in the art.

These screening methods enable the selection of drugs relating to the expression of the indicator gene in various ways. Specifically, for example, drug candidate compounds having the following activity can be found:

reduce the transcriptional activity of the indicator gene;

reduce the translation level from the transcript of the indicator gene;

inhibit the activity of translation product of the indicator gene; and

reduce the stability of the transcript of the indicator gene or accelerate its decomposition.

The above-described screening method of the present invention also includes a method comprising the step of stimulating test animals with an allergen before and/or after the administration of a candidate compound. When the allergen stimulation is performed prior to the administration of a candidate compound, it is possible to detect the activity of a candidate compound that inhibits immune response occurring after the allergen stimulation. Compounds obtainable by this screening method are expected to have therapeutic effects on an allergic disease. When allergen stimulation is conducted after the candidate compound administration, it is possible to detect the activity of a candidate compound that suppresses initiation of immune response occurring by the allergen stimulation. Compounds obtainable by this screening method are expected to have prophylactic effects on an allergic disease. An allergen usable in the screening method of this invention includes allergenic substances known in the art. More specifically, allergenic substances well-known in the art include mite, house dust, plant pollen, proteins derived from diverse foods, etc. as the These allergens may be derived from the nature or synthesized by gene recombination technique and the like method. Furthermore, allergens may be protein fragments. Methods for preparing a purified allergen are also well-known in the art.

For example, as a model closely resembling human atopic dermatitis, a spontaneous dermatitis model using NC/Nga mouse has been reported. Administration of the mite antigen (5 μg/ear) into the auricle of this mouse 8 times in total at 2 to 3 days intervals enables the induction of symptoms that closely resemble human atopic dermatitis after two weeks. The screening according to this invention can be conducted by administering a candidate compound to this system and monitoring changes in the expression level of the indicator gene of this invention.

In vitro screening can be performed, for example, by a method in which a candidate compound is contacted with cells expressing the indicator gene to select compounds that reduce the expression level of the indicator gene. The method may be carried out, for example, by the following steps of:

(a) contacting a candidate compound with cells that express the indicator gene;

(b) measuring the expression level of the indicator gene; and

(c) selecting the compound that reduces the expression level of the indicator gene, compared to a control with which the compound has not been contacted.

Peripheral blood leukocytes and cell lines derived from leukocytes can be used as indicator gene-expressing cells. Specifically, T cells can be exemplified as the leukocytes. T cell lines include Molt4 cells and Jurkat cells. Human acute leukemia T cell line Jurkat (ATCC Number TIB-152) can be obtained from ATCC.

In the screening method of the present invention, first a candidate compound is added to the cell. Then, the expression level of the indicator gene in the cell is measured to select the compound that reduces the expression level of the gene compared to that of a cell which has not been contacted with the candidate compound (control).

In the screening method of the present invention, expression level of the indicator gene can be compared by detecting the mRNA level transcribed from the gene or protein level encoded by the gene. When comparing the expression level using mRNA, an mRNA sample is prepared from the cells as described above. When comparing the expression level using protein, a protein sample is prepared from cells. Detection of mRNA and protein can be performed by known methods as described above.

Furthermore, based on the disclosure of this invention, it is possible to obtain the transcriptional regulatory region for the indicator gene of this invention to construct a reporter assay system. A reporter assay system means a assay system for screening for compounds that regulate the transcriptional activity of a transcriptional regulatory region using the expression level of a reporter gene localized downstream of the transcriptional regulatory region as an indicator. Specifically, such screening comprises the steps of:

(a) contacting a candidate compound with cells comprising a reporter gene that is linked to function under the control of a transcriptional regulatory region of the indicator gene;

(b) measuring the expression level of the reporter gene; and

(c) selecting the compound that decreases the expression level of the reporter gene, compared to a control with which the candidate compound has not been contacted.

The DNA region of about 0.5 kb to about 5 kb upstream from the transcription initiation point can be used as a transcriptional regulatory region. Transcriptional regulatory regions may include known transcriptional factor binding sequences, transcriptional regulating elements, and furthermore, CAAT box and TATA box, which are normally seen in the promoter region. Examples of the reporter genes include chloramphenicol acetyltransferase (CAT) gene, luciferase gene, growth hormone genes. The expression level of these reporter genes may be measured by detecting the activity of the expressed protein. Methods for detecting the activity of the proteins are well known in the art. A candidate compound that decreases the expression level of the reporter gene compared to that in a cell without having been contacted with the candidate compound, is selected.

The genomic sequence encoding the human B1799 gene (GenBank Ac. No. AL139230) has been revealed. Based on the sequence, the transcription initiation point and the upstream promoter sequence can be determined. A reporter construct can be prepared by amplifying the promoter region of B1799 gene utilizing PCR and such based on the sequence information, and linking a reporter gene downstream of the promoter region. Specifically, for example, a transcriptional regulatory region used for the screening of the present invention can be obtained as follows. First, screening is performed by a method that uses PCR or hybridization based on the nucleotide sequence of the indicator gene disclosed in the present invention or the sequence of the promoter region of the B1799 gene, to obtain a genomic DNA clone containing the cDNA sequence from a human genome DNA library, such as BAC library and YAC library. Based on the obtained genomic DNA sequence, the transcriptional regulatory region is obtained from upstream of the cDNA disclosed in the present invention. A reporter construct is constructed by cloning the obtained transcriptional regulatory region so that it is positioned upstream of the reporter gene. The resulting reporter construct is transformed into a cultured cell strain to prepare a transformant for screening. By contacting candidate compounds with this transformant, compounds that regulate the expression of reporter genes can be screened.

Furthermore, cells containing a reporter gene linked so as to function under the control of the transcriptional regulatory region of the indicator gene may be cells of so-called knockin animal. Knockin animal refers to a transgenic animal in which an exogenous gene has been inserted into the protein-coding region of the endogenous target gene. Since this knocked-in exogenous gene is inserted downstream of the transcriptional regulatory region of the target gene, it is expressed under a similar expression control to that for the endogenous target gene. It is possible to carry out the screening using the knockin animal or cells obtained from it using the reporter gene as a knockin gene. A screening method using a knockin animal comprises the steps of: (a) administering a candidate compound to an animal in which a reporter gene has been knocked-in to the indicator gene site, (b) measuring the expression level of the reporter gene, and (c) selecting a compound capable of reducing the expression level of the reporter gene compared to a control in which the candidate compound has not been administered. For example, the expression level of the reporter gene in leukocytes, more preferably in T cells of the knockin animal to which a candidate compound is administered or not administered, are measured to select a compound capable of reducing the expression level. Furthermore, a screening method using cells from the knockin animal comprises the steps of: (a) contacting a candidate compound with cells from an animal in which a reporter gene has been knocked-in to the indicator gene site, (b) measuring the expression level of the reporter gene, and (c) selecting a compound capable of reducing the expression level of the reporter gene compared to that in a control in which the candidate compound has not been contacted. For example, the expression level of the reporter gene in leukocytes, more preferably in T cells obtained from a knockin animal are measured in the presence or absence of a candidate compound to select a compound capable of reducing the expression level. These screenings are included in the screening method of the present invention. In the screening using an individual animal in particular, it is preferable to perform the screening using a knockin heterozygote in which one allele is left intact so as to express the indicator gene, while the reporter gene has been knocked in the other allele, to prevent the complete deletion of the function intrinsic to the indicator gene.

Alternatively, screening based on the activity of the indicator protein can be employed as the in vitro screening method of the present invention. More specifically, the present invention relates to a method of screening for therapeutic agents for allergic diseases comprising the steps of:

(a) contacting a candidate compound with a protein encoded by the indicator gene of the present invention or a gene functionally equivalent thereto;

(b) measuring the activity of the protein; and

(c) selecting the compound that reduces the activity of the protein as compared to a control with which the candidate compound has not been contacted.

Herein, genes functionally equivalent to the indicator gene include endogenous B1799 gene that has been artificially modified as described above.

Such screening can be conducted, for example, by allowing host cells to exogenously express the indicator gene and measuring the activity of the indicator protein. In this case, the indicator gene may be inserted into an expression vector, and the resulting recombinant vector is introduced into appropriate hosts such as mammalian cells. The vectors can be introduced into the host by, for example, biological, physical, or chemical methods. Examples of the biological method are a method using viral vectors, a method using a specific receptor, and cell fusion method (via HVJ (Sendai virus), polyethylene glycol (PEG) method, electric cell fusion method, and microcell-mediated chromosome transfer). Examples of the physical method are a microinjection method, electroporation method, and a method using the gene particle gun (gene gun). Examples of the chemical method are a calcium phosphate precipitation method, liposome method, DEAE-dextran method, protoplast method, red cell ghost method, red cell membrane ghost method, and microcapsule method.

Activities of the indicator protein measured in the screening include, for example, binding activity to GTP binding proteins or GAP activity. Furthermore, for example, the indicator protein is suggested to be related to the regulation of intracellular signal transduction in T cells, and the regulation of differentiation or proliferation of T cells. Therefore, using these activities as an index, compounds having an activity to inhibit these activities may be screened. Such compounds obtained by the method repress the function of the indicator protein. As a result, allergic immune response can be suppressed through the inhibition of the indicator protein activity that is induced in the T cells.

The polynucleotide, antibody, cell line, or animal model required for the various screening methods of the present invention may be previously combined into a kit. More specifically, a kit is composed of, for example, a cell (cultured cell, animal, or such) expressing the indicator gene, and a reagent for measuring the expression level of the indicator gene. Reagents used for measuring the expression level of the indicator gene include, for example, oligonucleotides comprising at least 15 continuous nucleotides of the nucleotide sequence of the indicator gene or the complementary sequence thereof. Alternatively, antibodies binding to polypeptides comprising the amino acid sequence of the indicator protein can be used as the reagent. In the kit may be packaged a substrate compound used for the detection of a label, medium and a container for cell culturing, positive and negative standard samples, and furthermore, a manual describing how to use the kit.

Candidate compounds used in these screening include compound preparations, such as immunosuppressors, synthesized by existing chemical methods, compound preparations synthesized by combinatorial chemistry, mixtures of multiple compounds such as extracts from animal or plant tissues, or microbial cultures, and their purified preparations.

Compounds selected by the screening method of the present invention are useful as a therapeutic agent for an allergic disease. Alternatively, antisense DNA capable of inhibiting the expression of the indicator gene in this invention is useful for this purpose. Such antisense DNAs are those comprising sequences complementary to the sequences containing preferably at least 20, more preferably 25, 30, 40, 50, and 100 or more continuous nucleotides of the sense strand of the indicator gene of this invention. The antisense region may be antisense to any region of transcripts (any transcripts before and after the processing, including the intermediary product) of the indicator gene. One example of such region is that containing the translation initiation codon. Furthermore, an antibody binding to the protein encoded by the indicator gene used in the present invention is useful as a therapeutic agent for an allergic disease. Preferable antibodies are those binding to the domain interacting with a GTP binding protein, such as, antibodies to the TBC domain (the amino acid region from position 965 to 1184 of SEQ ID NO: 2 or homologous region thereto), specifically antibodies that identify as an epitope a peptide comprising the Arg residue (e.g., the position 973 Arg residue of SEQ ID NO:2) necessary as the GTPase activating site, are preferred. Antibodies against a phosphotyrosine interaction domain (PID) (the amino acid region from position 167 to 237 or from 415 to 487 of SEQ ID NO: 2, or homologous region thereto) are also preferred. The therapeutic agent for an allergic disease according to the present invention contains a compound selected by the screening method, the antisense DNA, or the antibody as at least one main ingredient, and can be prepared by mixing the ingredient with a physiologically acceptable carrier, excipient, diluent, etc. The therapeutic agent for an allergic disease according to this invention can be administered orally or parenterally to ameliorate allergic symptoms.

For an oral drug, the dosage form can be granules, powder, tablets, capsules, solution, emulsion, suspension, etc. Examples of injections are subcutaneous, intramuscular and peritoneal injections.

Furthermore, in the case where a compound to be administered is a protein, the therapeutic effect can be achieved by introducing a gene encoding the protein into the living body using a gene therapy technique. A technique for treating a disease by introducing a gene into the living body that encodes a protein having a therapeutic effect is well-known in the art.

Alternatively, an antisense DNA can be directly administered. In that case, cell membrane permeability or stability of the DNA can be elevated by, for example, 5′ end and/or 3′ end modification. Alternatively, the antisense DNA may be incorporated downstream of an appropriate promoter sequence to be administered as an antisense RNA expression vector. When this expression vector is introduced into T cells of an allergic disease patient, the expression level of the indicator gene can be reduced due to the expression of antisense of the gene, thereby achieving a therapeutic effect on an allergic disease. For introducing the expression vector into T cells, methods performed either in vivo or ex vivo are known.

Although the dosage of the therapeutic agent for an allergic disease according to the invention may vary depending on the age, sex, body weight, and symptoms of a patient; therapeutic effects; methods for administration; treatment duration; types of active ingredient contained in the pharmaceutical composition or such, it can be usually administered in the range of 0.1 mg to 500 mg, preferably 0.5 mg to 20 mg per dose for an adult. However, since the dosage varies according to various conditions, an amount less than the above-described dosage may be sufficient in some cases, and a dosage exceeding the above-described range may be required in other cases.

The present invention provided a gene that shows a difference in expression between healthy subjects and patients with allergic diseases. Using the expression of the gene of the present invention as an indicator, it became possible to test for allergic diseases and screen for candidate therapeutic compounds. The test of the present invention can be readily conducted by measuring the expression level of the indicator gene and can quickly find the pathological state of allergic reaction. Additionally, according to this invention, the method of testing for allergies has low invasiveness towards patients since analysis of the expression level can be carried out using biological samples, like peripheral blood leukocytes. Furthermore, highly sensitive measurements in gene expression analysis are possible using small sample amounts. Year after year, high throughput methods and cost reduction are progressing in gene analysis technology. Therefore, in the near future, the method of testing for allergies of this invention is expected to be come an important bed-side diagnostic method. In this sense, the diagnostic value of these pathology-related genes is high. Furthermore, the screening methods of the present invention are expected to be applied to development of novel therapeutic agents for allergic diseases.

The present invention will be explained in detail below with reference to examples, but it is not to be construed as being limited thereto.

### EXAMPLE 1

### Identification of B1799

The Differential Display (DD) method (Liang and Pardee, Science, 1992, 257: 967-971; T. Ito et al., 1994, FEBS Lett. 351: 231-236) discovered a 164 bp DNA fragment (clone B1799-01) (SEQ ID NO: 3; except for primer sequence) that is expressed much stronger in T cells of atopic dermatitis patients and allergic asthma patients compared to that of healthy subjects. For the DD analysis to detect the fragment, GT15C (SEQ ID NO: 5) and AG00189 (TCTCTGGAGT; SEQ ID NO: 6) were used as anchor primer and arbitrary primer, respectively. The gene from which the fragment was derived was designated as B1799.

### EXAMPLE 2

### Cloning of B1799

PCR cloning was performed based on the 5′-RACE method using the DD sequence and as a template a human leukocyte cDNA library to obtain a DNA fragment of a full length of 486 bp (SEQ ID NO: 4). BLAST search on a public database revealed that the sequence at the position 27 to 255 of this nucleotide sequence was 100% identical to the gene KIAA0603 (GenBank Accession No. AB011175) that was identified by Kazusa DNA Research Institute. KIAA0603 is a cDNA of 5922 bp that encodes a protein consisting of 1299 amino acid residues starting with methionine in its ORF. The homologous sequence region was within the ORF region, i.e., from nt 2152 to nt 2380, of KIAA0603 gene. Furthermore, 486 bp sequence was completely identical with the nt 128071 to nt 127586 region (reverse strand) of GenBank Accession No. AL162571 that is a genomic sequence of chromosome 13.

### EXAMPLE 3

### Expression Quantification of B1799 by Quantitative PCR

The DD sequence was determined to be derived from the immature mRNA intron region of KIAA0603 gene. Assuming that B1799 is identical to KIAA0603, KIAA0603 gene was analyzed. The following primers and probes for mRNA quantification were designed within the ORF of KIAA0603 gene and used for expression quantification. TQ1799orf is a TaqMan probe used in gene expression quantification by TaqMan method, whose 5′ end and 3′ end are fluorescent labeled with FAM (6-carboxyfluorescein) and TAMRA (6-carboxy-methyl-rhodamine), respectively. Using ABI-PRISM 7700, DNA amplification was detected at real time.

| | | |
|---|---|---|
| 1799orf-f: | AAGACAGTGGAGCAACTCCGG | (SEQ ID NO: 7) |
| 1799orf-r: | CAGCAACAGGTCACAATTGGC | (SEQ ID NO: 8) |
| TQ1799orf: | AGCTGCTGCCCGCGGATGCT | (SEQ ID NO: 9) |

### EXAMPLE 4

### Relevance to Pathology

The expression of B1799 gene was measured using RNAs prepared from T cells of atopic dermatitis patients as described in Example 3. The results are shown in Table 1. According to a t-test of the subjects divided into two groups, i.e., healthy subject group and atopic dermatitis patient group, the expression level was significantly higher (p<0.05) in the patient group.

TABLE 1
Expression level oif B1799 in atopic dermatitis patients

| | | B1799 (copy/ng | | | B1799 (copy/ng |
|---|---|---|---|---|---|
| | Pathology | RNA) | | Pathology | RNA) |
| 1 | normal | 5196.77 | 21 | moderate | 3865.66 |
| 2 | normal | 2813.08 | 22 | moderate | 3931.79 |
| 3 | normal | 4443.58 | 23 | moderate | 6614.39 |
| 4 | normal | 4115.51 | 24 | moderate | 6352.02 |
| 5 | normal | 3890.60 | 25 | moderate | 15627.77 |
| 6 | normal | 1897.45 | 26 | moderate | 4516.00 |
| 7 | normal | 2163.33 | 27 | moderate | 8289.22 |
| 8 | normal | 154.31 | 28 | moderate | 17001.41 |
| 9 | normal | 3542.13 | 29 | moderate | 5502.07 |
| 10 | normal | 1647.13 | 30 | moderate | 21914.55 |
| 11 | mild | 1176.89 | 31 | severe | 2645.44 |
| 12 | mild | 10222.95 | 32 | severe | 3492.70 |
| 13 | mild | 15579.36 | 33 | severe | 5810.25 |
| 14 | mild | 18515.82 | 34 | severe | 13260.42 |
| 15 | mild | 3200.39 | 35 | severe | 736.77 |
| 16 | mild | 1862.32 | 36 | severe | 6562.17 |
| 17 | mild | 5031.60 | 37 | severe | 11227.19 |
| 18 | mild | 5999.48 | 38 | severe | 5974.82 |
| 19 | mild | 5382.48 | 39 | severe | 3249.61 |
| 20 | mild | 1019.33 | 40 | severe | 609.08 |

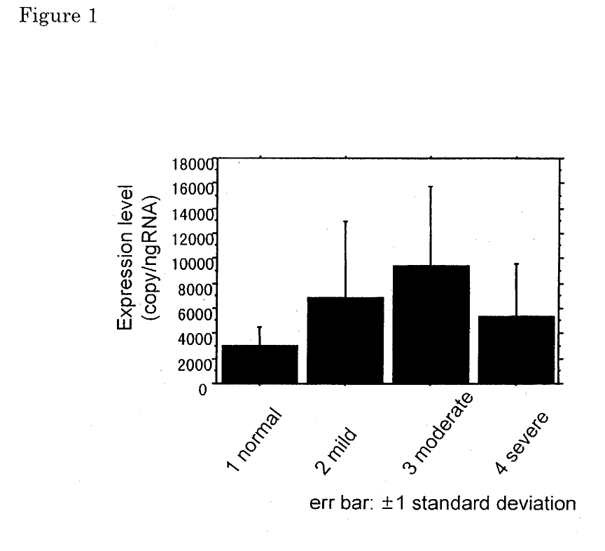

Specifically, the expression level was high in patients with moderate pathology compared to healthy subjects and patients with mild pathology. More specifically, analysis of variance was performed among four groups consisting of groups of atopic dermatitis classified into groups of mild, moderate, and severe pathologies, and the healthy subject group, indicating that there are significant differences (p<0.05) among these four groups. Comparison of the differences among the groups by post hoc test according to the PLSD method of Fisher, showed the significant difference and bias between the groups as follows. Expression in each group is shown in FIG. 1.

healthy subject < mild pahology subject

(bias p < 0.1)

healthy subject < moderate pathology subject

(significant difference p < 0.01)

severe pathology subject < moderate pathology subject

(bias p < 0.1)

As demonstrated above, the expression level was revealed to be higher in atopic dermatitis patients, especially in patients with moderate pathology, compared to normal healthy subjects.

### EXAMPLE 5

### Expression Levels in Various Immunocytes

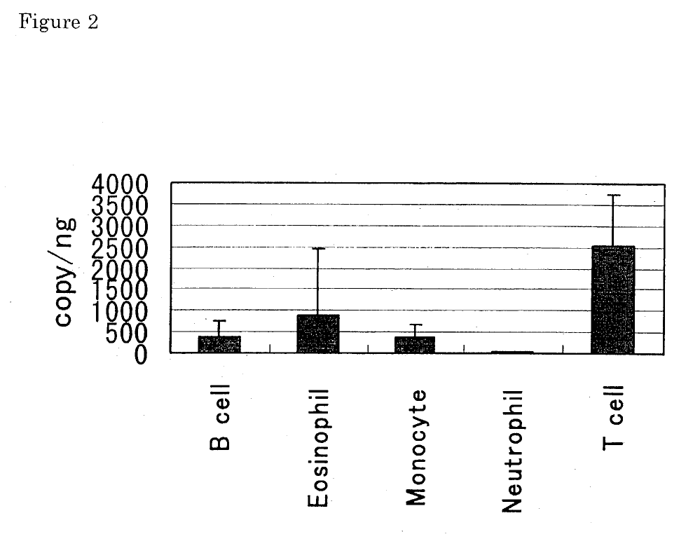

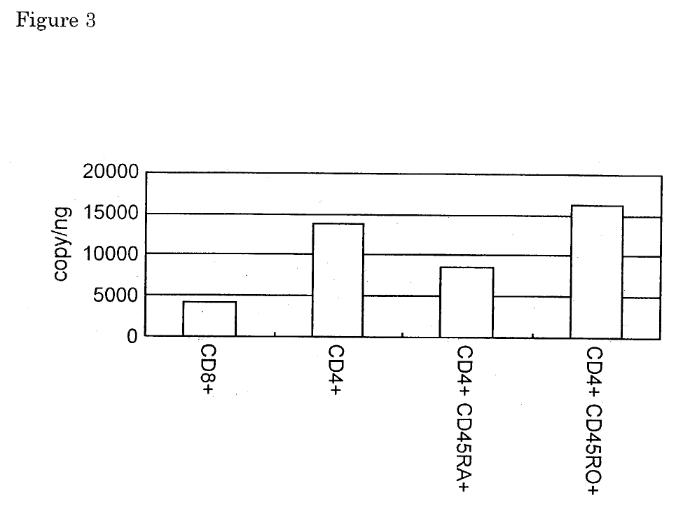

Expression levels in T cells, B cells, eosinophils, monocytes, and neutrophils were measured in RNA samples derived from 5 subjects. The result revealed that the expression level was highest in T cells (FIG. 2). Among the T cells, the expression was higher in T cells with the phenotypes of CD4⁺ and CD45RO⁺, i.e., memory T cells (FIG. 3).

### EXAMPLE 6

### Changes in Expression Due to T Cell Activation

Once mature T cells are stimulated with antigen, a further stimulation with the antigen is known to cause apoptosis which is called activated cell death of T cells. Activated cell death of T cells is suggested to be involved in the control of immunological tolerance and immune response in periphery (Yili Yang et al., J. Exp. Med. 181: 1673-1682, 1995). To examine the role of B1799 in these controls, the expression changes of B1799 due to activated cell death of T cells and cell death caused by glucocorticoid were measured. Peripheral blood-derived T cells were cultured in 5% FCS-containing RPMI1640 medium supplemented with 1 mM sodium pyruvate, 2 mM L-glutamine, 100 u/ml penicillin, and 100 μg/ml streptomycin, further with 200 u/ml IL-2 (Imunace®, Shionogi & Co., Ltd.) under 5% carbon dioxide and 95% humidity at 37° C. First, CD3+ cells isolated from periphery were cultured for 5 days on plates coated with an anti-CD3 antibody (OKT3, Janssen-Kyowa Co., LTD), followed by cultivation for 3 days without the anti-CD3 antibody. The obtained T cells were cultured again with stimulus on the anti-CD3 antibody coated plates to measure activated cell death. Cells are collected over the course of time, and RNA was extracted according to conventional methods for quantitative PCR. For control without stimulus, plates without an anti-CD3 antibody were used.

Further, dexamethasone was added to unstimulated control as glucocorticoid that induces apoptosis of T cells (Kofler R., Histochem. Cell Biol., 2000 July; 114(1): 1-7), and its influence was examined.

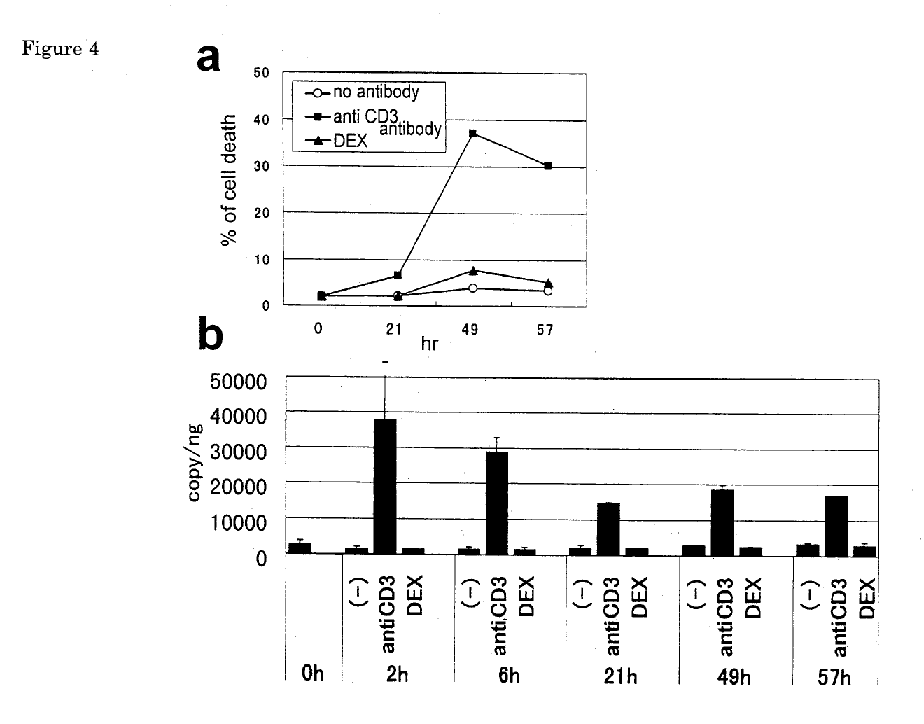

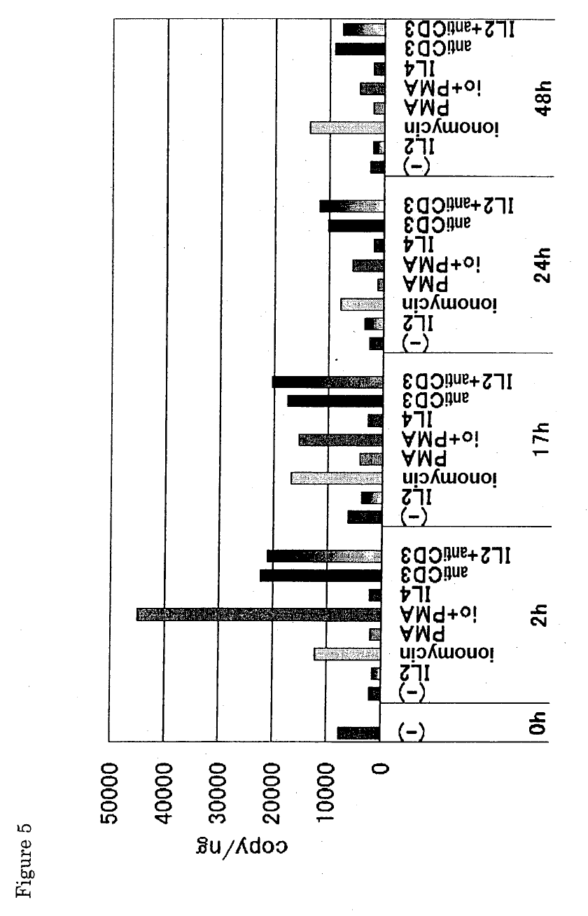

As depicted in FIG. 4a, obvious cell death was induced 49 hours after the second stimulation via the T cell receptor on human periphery-derived T cells using anti-CD3 antibodies. B1799 was markedly induced 2 hours after the stimulation during the T cell activation process. In contrast, no notable cell death of peripheral T cells or changes in the expression of B1799 was observed in the presence of dexamethasone (FIG. 4b “DEX”). Through the stimulation of T cells by various reagents in similar experiments, the expression of B1799 was shown to be markedly induced by stimulation with 1 μg/ml of ionomycin (Sigma), calcium ionophore, alone (described as “ionomycin” in the Figure), or with 25 ng/ml of phorbol 12-myristate 13-acetate (PMA, Sigma) (described as “io+PMA” in the Figure) (FIG. 5), in addition to anti-CD3 antibodies. The above results show that the expression of B1799 was enhanced together with the activation of the T cell. Furthermore, B1799 was suggested to be involved in some way to subsequent activated cell death.

### EXAMPLE 7

### Analysis of Amino Acid Sequence Structure

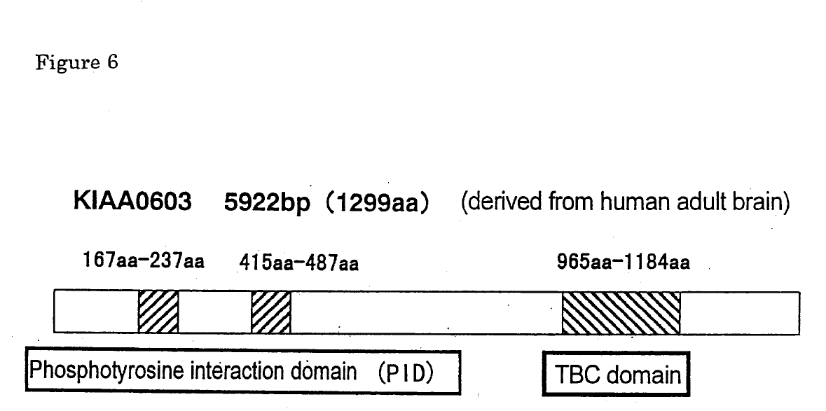

The structure of the protein encoded by B1799 is shown in FIG. 6. The protein has, at its N-terminus region, two phosphotyrosine interaction domain (PID) that is involved in the inter-protein binding in the phosphorylated tyrosine binding region. Furthermore, the protein has a TBC domain at its C-terminus.

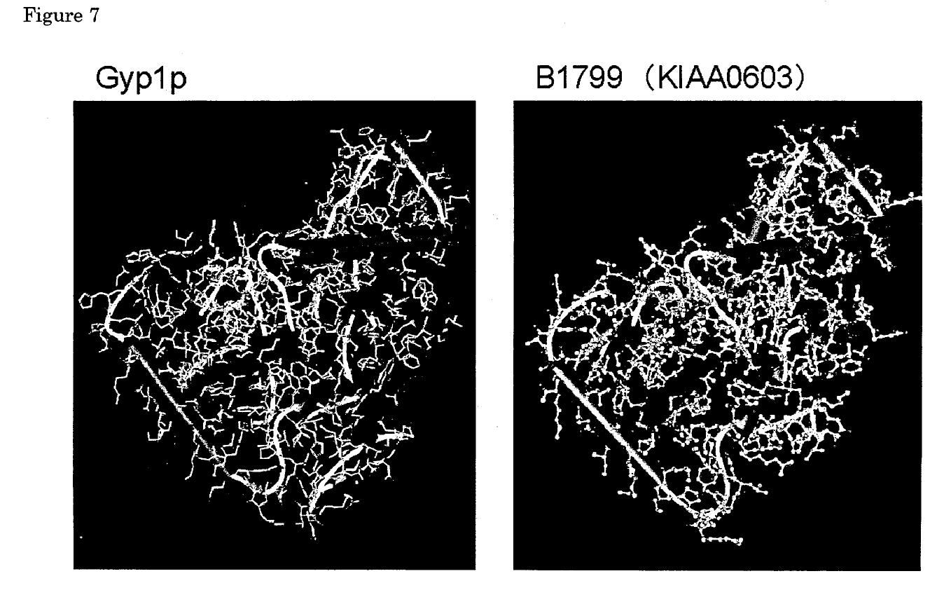

BLAST search revealed that the region of amino acids from the position 886 to 1237, which contains the TBC domain, shows a homology of 31% to human rab6 GTPase activating protein (Cuif M. H. et al., EMBO J. :18(7) 1772-82, 1999) (ACCESSION NP_{—}036329). Further, BLAST search showed that the amino acid region from the position 853 to 1107 has a homology of 21% to Gyp1p protein (ACCESSION NP_{—}014713), yeast GTPase activating protein (GAP). As demonstrated in FIG. 7, B1799 protein was predicted to have a very similar tertiary structure to the GTPase activating site structure of Gyp1, whose crystal structure had been determined (Rak A. et al., EMBO J. 19: 5105-5113, 2000). Arg973 residue of B1799 protein corresponds to Arg343 residue of Gyp1p, which is essential as the GTPase activating site. Therefore, there is a high possibility that the B1799 protein is a GTPase activating protein, and the Arg973 residue was predicted to play an important role in its activity.

The GTP binding form of the GTP binding protein, which protein has an important role in the signal transduction, is the activated form in the signal transduction and changes to the inactivated form when the bound GTP is converted to GDP. The GTP binding protein itself has the GTPase activity and GTPase activating proteins have the function to enhance the GTPase activity. GTPase activating proteins help the conversion of GTP binding proteins to the inactivated form, thereby regulating signal transduction.

As described above, B1799 would play an important role in signal transduction where the T cell gets activated by regulating the activity of the GTP binding protein.

Furthermore, throughout the whole sequence, the amino acid sequence encoded by B1799 showed a high homology to mouse Tbc1. Tbc1 was discovered as a gene whose expression changes during mast cell differentiation, and is suggested to be involved in cell differentiation and cell cycle suppression (Paul M. Richardson and Leonard I. Zon., Oncogene 11: 1139-1148, 1995). Thus, similarly to Tbc1, B1799 maybe involved in cell differentiation and cell cycle suppression.

What is claimed is:

1. A method of testing for an allergic disease, the method comprising the steps of:
(a) measuring the expression level of an indicator gene in a biological sample from a test subject;
(b) comparing the expression level with that of the indicator gene in a biological sample from a healthy subject; and
(c) judging the test subject to have an allergic disease when the expression level of the indicator gene in the biological sample from the test subject is found to be significantly elevated. wherein the indicator gene is B1799 gene.

2. The testing method according to claim 1, wherein the allergic disease is atopic dermatitis.

3. The testing method according to claim 1, wherein the gene expression level is measured via cDNA PCR.

4. The testing method according to claim 1, wherein the gene expression level is measured by detecting a protein encoded by the gene.

5. The testing method according to claim 1, wherein the biological sample contains peripheral blood T cells.

6. A reagent for diagnosis of an allergic disease, said reagent comprising a polynucleotide that comprises at least 15 continuous nucleotide sequence of B1799 gene or a complementary sequence thereof;

7. A reagent for testing for an allergic disease, said reagent comprising an antibody that binds to a polypeptide consisting of the amino acid sequence encoded by B1799 gene;

8. A method for screening a therapeutic agent for an allergic disease, wherein said method comprises the steps of:
(a) contacting a candidate compound with a cell expressing an indicator gene;
(b) measuring the expression level of said indicator gene; and
(c) selecting a compound which decreases the expression level of the indicator gene as compared to a control where said candidate compound has not been contacted,
wherein the indicator gene is B1799 gene.

9. The method according to claim 8, wherein the cell is T cell.

10. A method of screening for a therapeutic agent for an allergic disease, the method comprising the steps of:
(a) administering a candidate compound to a test animal;
(b) measuring the expression level of an indicator gene in leukocytes of the test animal; and
(c) selecting a compound which decreases the expression level of the indicator gene compared to a control where the candidate compound has not been administered,
wherein the indicator gene is B1799 gene.

11. A method of screening for a therapeutic agent for an allergic disease, the method comprising the steps of:
(a) contacting a candidate compound with cells containing a reporter gene linked under the control of transcriptional regulatory region of an indicator gene;
(b) measuring the expression level of the reporter gene; and
(c) selecting a compound which decreases the expression level of the reporter gene compared to a control where the candidate compound has not been contacted,
wherein the indicator gene is B1799 gene.

12. A method of screening for a therapeutic agent for an allergic disease, the method comprising the steps of:
(a) contacting a candidate compound with a protein encoded by an indicator gene or a gene functionally equivalent thereto;
(b) measuring the activity of the protein; and
(c) selecting a compound which decreases the activity of the protein compared to a control where the candidate compound has not been contacted,
wherein the indicator gene is B1799 gene.

13. A therapeutic agent for an allergic disease comprising as the main ingredient a compound obtainable by the screening method according to any one of claim 8, 10, 11, and 12.

14. A therapeutic agent for an allergic disease, which comprises as a main ingredient an antisense DNA that contains a sequence complementary to a sequence comprising at least 15 continuous nucleotides of the sense strand sequence of an indicator gene, wherein the indicator gene is B1799 gene.

15. A therapeutic agent for an allergic disease, which comprises as a main ingredient an antibody which binds to a protein encoded by an indicator gene, wherein the indicator gene is B1799 gene.

16. An animal model of allergic disease, wherein said animal is a transgenic nonhuman vertebrate, in which the expression level of an indicator gene, or a gene functionally equivalent thereto, has been increased in T cells, wherein the indicator gene is B1799 gene.

17. A kit for screening for a therapeutic agent for an allergic disease, the kit comprising a polynucleotide comprising at least 15 continuous nucleotide sequence of an indicator gene or its complementary sequence, wherein the indicator gene is B1799 gene.

18. A kit for screening for a therapeutic agent for an allergic disease, the kit comprising an antibody which binds to a polypeptide consisting an amino acid sequence encoded by an indicator gene and cells expressing the indicator gene, wherein the indicator gene is B1799 gene.
